(19) United States
(12) Patent Application Publication
(10) Pub. No.: US 2026/0037618 A1
(43) Pub. Date: Feb. 5, 2026

(54) APPARATUS AND METHOD FOR VERIFYING FORGERY AND TAMPERING OF MEDICAL IMAGE

(71) Applicant: ICERTI CO., LTD, Daejeon (KR)

(72) Inventors: Young Hu KIM, Seoul (KR); Sang Kwon LEE, Seoul (KR); Hyun Su KIM, Seoul (KR); Hyun Gi KIM, Seoul (KR)

(21) Appl. No.: 19/291,466

(22) Filed: Aug. 5, 2025

(30) Foreign Application Priority Data
Aug. 5, 2024 (KR) 10-2024-0103854

(51) Int. Cl. G06F 21/55; G06F 21/62; G16H 30/20

(52) U.S. Cl. CPC G06F 21/55; G06F 21/6245; G16H 30/20; G06F 2221/034

(57) ABSTRACT

Provided are an apparatus and method for verifying forgery and tampering of a medical image. An apparatus for verifying forgery and tampering of a medical image according to an embodiment disclosed in this document includes a memory configured to store Digital Imaging and Communications in Medicine (DICOM) files for each patient, and a processor functionally connected to the memory, wherein, when an issuance request of a DICOM file is received, search for a DICOM file corresponding to the request among DICOM files for each patient; generate an issuance number related to the issuance; extract some information from metadata of the searched DICOM file; generate a first hash value using the some information and the issuance number; and upon providing a copy of the DICOM file to an external storage device, insert the first hash value as forgery verification information into a specified tag area of the copy.

### CROSS-REFERENCE TO RELATED APPLICATION

This application claims priority to and the benefit of Korean Patent Application No. 10-2024-0103854, filed on Aug. 5, 2024, the disclosure of which is incorporated herein by reference in its entirety.

### BACKGROUND

### 1. Field of the Invention

Various embodiments disclosed in this document relate to a technique for verifying forgery and tampering of data.

### 2. Discussion of Related Art

Clinical diagnosis plays a major role in patient treatment, and dependence on it is expected to increase in the future. Clinical diagnosis is performed using various medical image devices such as X-ray, computed tomography (CT), or magnetic resonance imaging (MRI) devices.

Patients may obtain their own clinical diagnosis data by proving their identity as necessary (e.g., when changing hospitals or proving illness). For example, a patient may obtain medical image data from a hospital to share a treatment status or provide proof of medical history. Usually, a hospital issues a medical image compact disc (CD) after verifying the identity of a patient who has visited the hospital.

### SUMMARY OF THE INVENTION

However, medical image compact discs (CDs) are not forgery-proof, and thus there is no way to check forgery of medical image data even when submitting medical image data of others for exemption from military service. Furthermore, some hospitals send medical image data by mail, but there is a risk of loss or duplication during delivery as the medical image data is processed as regular mail.

Various embodiments disclosed in this document are directed to providing an apparatus and method for verifying forgery and tampering of a medical image that are capable of verifying forgery and tampering of the medical image using a hash value based on metadata of the medical image.

An apparatus for verifying forgery and tampering of a medical image according to an embodiment disclosed in this document includes a memory configured to store Digital Imaging and Communications in Medicine (DICOM) files for each patient, and a processor functionally connected to the memory, wherein, when an issuance request of a DICOM file is received, search for a DICOM file corresponding to the request among DICOM files for each patient; generate an issuance number related to the issuance; extract some information from metadata of the searched DICOM file; generate a first hash value using the some information and the issuance number; and upon providing a copy of the DICOM file to an external storage device, insert the first hash value as forgery verification information into a specified tag area of the copy.

Further, an apparatus for verifying forgery and tampering of a medical image according to an embodiment disclosed in this document includes a memory configured to store verification data including issuance numbers of Digital Imaging and Communications in Medicine (DICOM) files for each patient and hash values of each DICOM files; and a processor functionally connected to the memory, wherein, when obtaining a request for verification of a first DICOM file stored in an external storage device, the processor extracts a first hash value and first issuance number from a specified tag area of the first DICOM file, generates a first hash value to be verified using some information of metadata of the first DICOM file, and performs primary verification of whether the first DICOM file has been forged by comparing the extracted first hash value with the first hash value to be verified.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 illustrates a configuration diagram of a system for verifying forgery and tampering of a medical image according to an embodiment;

FIG. 2 illustrates a configuration diagram of an apparatus for verifying forgery and tampering of a medical image according to an embodiment;

FIG. 3 illustrates an example in which a Digital Imaging and Communications in Medicine (DICOM) file according to an embodiment is used;

FIG. 4 illustrates a flowchart of a method of verifying forgery and tampering of a medical image according to an embodiment;

FIG. 5 illustrates a flowchart of a method of verifying forgery of a medical image according to an embodiment; and

FIG. 6 illustrates an example in which a result of verification according to an embodiment is provided.

With respect to the descriptions of the drawings, the same or similar reference numerals may be used for the same or similar elements.

### DETAILED DESCRIPTION OF EXEMPLARY EMBODIMENTS

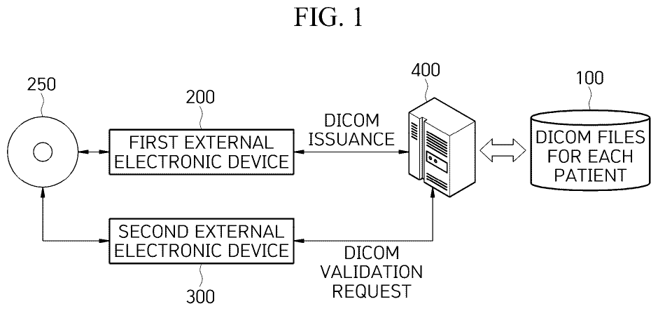

FIG. 1 illustrates a configuration diagram of a system for verifying forgery and tampering of a medical image according to an embodiment.

Referring to FIG. 1, the system for verifying forgery and tampering of the medical image according to an embodiment may include a database (DB) 100, an external storage device 250, a first external electronic device 200, a second external electronic device 300, and an apparatus 400 for verifying forgery and tampering (Hereinafter, referred to ‘apparatus 400 for verifying forgery & tampering’).

According to an embodiment, the DB 100 is a DB that stores medical data and may store a Digital Imaging and Communications in Medicine (DICOM) file for each patient in relation to patient information. The DICOM file may be a file with the extension “*.DCM” according to the DICOM international agreement. Further, the DB 100 may store verification data related to verification of forgery of issued DICOM files.

According to an embodiment, the DB 100 may include a first storage device that stores medical data and a second storage device that stores verification data. The first and second storage devices may be different devices or may be included in different devices. Further, the DB 100 may be included in the apparatus 400 for verifying forgery & tampering.

According to an embodiment, the external storage device 250 may include at least one of a compact disc (CD) for recording medical data (DICOM files) to be issued and a memory connected to the first external electronic device 200. The external storage device 250 may store a DICOM file into which forgery verification information is inserted under the control of the first external electronic device 200 or the apparatus 400 for verifying forgery & tampering.

According to an embodiment, the first external electronic device 200 may be a terminal of a first user who requests issuance of medical data. For example, the first external electronic device 200 may be a computing terminal used by medical personnel (e.g., an administrative staff member). The first external electronic device 200 may transmit a request for issuance of a DICOM file together with patient information input by the medical personnel to the apparatus 400 for verifying forgery & tampering. As another example, the first external electronic device 200 may be a computing terminal used by a subject (a target person of the DICOM file). In this case, the first external electronic device 200 may access a first website related to online issuance of medical data. The first external electronic device 200 may obtain the patient information input by the subject and transmit a request for issuance of a DICOM file including the patient information to the apparatus 400 for verifying forgery & tampering through the first website.

According to an embodiment, the first external electronic device 200 may store a DICOM file issued under the control of the apparatus 400 for verifying forgery & tampering in the external storage device 250. For example, when a DICOM file is issued online (e.g., issued through a homepage), the first external electronic device 200 may obtain a password from a second user and transmit the obtained password to the apparatus 400 for verifying forgery & tampering. In this case, the apparatus 400 for verifying forgery & tampering may encrypt and compress a DICOM file to be issued using the obtained password and generate an encrypted compressed file in the first external electronic device 200. The apparatus 400 for verifying forgery & tampering may, for example, encrypt and compress a DICOM file group including a plurality of DICOM files using one password and generate one DICOM file group as one compressed file. Thereafter, the first external electronic device 200 may download the encrypted compressed file from the apparatus 400 for verifying forgery & tampering and store (or record) the downloaded encrypted compressed file in the external storage device 250. The encrypted compressed file may be decrypted a password preregistered by a user is input. As another example, when a DICOM file is issued offline (e.g., issued at a medical institution by medical personnel), the first external electronic device 200 may store a DICOM file issued by the apparatus 400 for verifying forgery & tampering in the external storage device 250.

According to an embodiment, the second external electronic device 300 may be a terminal that requests verification of medical data. For example, the second external electronic device 300 may be a computing terminal used by a user involved in verification of medical data (e.g., a Military Manpower Administration employee). The second external electronic device 300 may access a second website involved in verification of medical data and transmit a request for forgery verification of the DICOM file to the apparatus 400 for verifying forgery & tampering through the second website. Thereafter, when the apparatus 400 for verifying forgery & tampering completes the forgery verification, the second external electronic device 300 may obtain a result of the forgery verification of the DICOM file and output the obtained result through a display. The first and second websites may be, for example, different web pages of the same site. Alternatively, the first and second websites may be a plurality of websites, each of which is specialized in issuance or verification.

First, a process of issuing a DICOM file performed by the apparatus 400 for verifying forgery & tampering will be described.

According to an embodiment, when the apparatus 400 for verifying forgery & tampering obtains a request for issuance of a DICOM file from the first external electronic device 200, the apparatus 400 for verifying forgery & tampering may search for a DICOM file group corresponding to the request for issuance among the DICOM files for each patient from the medical DB 100. For example, when first patient information (e.g., a resident registration number, a department of medicine, an examination date, etc.) is obtained through an input interface, the apparatus 400 for verifying forgery & tampering may search for a DICOM file corresponding to the first patient information from the DB 100.

According to an embodiment, the apparatus 400 for verifying forgery & tampering may generate a first issuance number related to a DICOM file to be issued and extract some information from metadata of the searched DICOM file group. The apparatus 400 for verifying forgery & tampering may generate a first hash value for primary verification using the extracted some information and the first issuance number. The some information may include, for example, at least one of the first patient information and issuer information (e.g., a hospital name, an issuance date, etc.) of the DICOM file group.

The apparatus 400 for verifying forgery & tampering may provide a copy of the DICOM file group to the external storage device 250 to insert the first hash value as forgery verification information into a specified tag area of the corresponding copy. The specified tag area may be, for example, a user-defined tag area (e.g., Ox0011) of the DICOM file. The apparatus 400 for verifying forgery & tampering may generate a second hash value for secondary verification on the basis of copies of the individual DICOM files of the DICOM file group.

The apparatus 400 for verifying forgery & tampering may store the second hash value in the DB 100 as verification data of the copy in relation to the first issuance number.

According to the above-described embodiment, a medical image may be composed of a DICOM file group including a plurality of DICOM files. Further, the first issuance number may include serial numbers of the respective DICOM files and an original number of the DICOM file group. In this case, the apparatus 400 for verifying forgery & tampering may calculate first hash values for the respective DICOM files on the basis of the serial numbers of the respective DICOM files and the original number. In this case, the respective DICOM files may have different forgery verification information (first hash values) inserted.

According to various embodiments, when DICOM files are issued online, the apparatus 400 for verifying forgery & tampering may obtain a password for compression from the first user, compress the DICOM files using the obtained password, and store (or record) a compressed file in the external storage device 250 through the first external electronic device 200.

According to various embodiments, the apparatus 400 for verifying forgery & tampering may calculate third hash values for the searched individual DICOM files (the original DICOM files) and calculate first hash values on the basis of the third hash values, the first issuance number, and the extracted some information. In this case, the apparatus 400 for verifying forgery & tampering may also store the third hash value in the specified tag area of the copy of the DICOM file.

Next, a process of verifying forgery of a DICOM file performed by the apparatus 400 for verifying forgery & tampering will be described.

According to an embodiment, the apparatus 400 for verifying forgery & tampering may obtain a request for forgery verification of a first DICOM file stored in the external storage device 250 from the second external electronic device 300. In this case, the apparatus 400 for verifying forgery & tampering may provide a first website (or application) related to the verification of the first DICOM file to the second external electronic device 300 through a web browser.

The second external electronic device 300 may access the first website and extract a first hash value and first issuance number from the specified tag area of the copy of the first DICOM file to be verified according to the execution of the web browser. Further, the second external electronic device 300 may generate a first hash value to be verified on the basis of some information among metadata of the first DICOM file stored in the external storage device 250. According to an embodiment, primary verification may be performed by comparing the first hash value to be verified with the extracted first hash value. When the first hash value to be verified matches the extracted first hash value, the second external electronic device 300 may notify the second external electronic device 300 of the completion of the primary verification of the first DICOM file. In this way, the apparatus 400 for verifying forgery & tampering according to an embodiment may provide verification of the first DICOM file on a browser basis, and thus the second external electronic device 300 may perform the primary verification on its own without transmitting or receiving data for verification of the first DICOM file to or from the apparatus 400 for verifying forgery & tampering. Therefore, in an embodiment, the primary verification for checking for forgery of DICOM files may be very rapidly performed, crude forgery at the level of simply exchanging DICOM files of other users may be rapidly filtered out, and secondary verification may be supported to be performed only on DICOM files that have been elaborately forged by experts.

On the other hand, when the first hash value to be verified does not match the extracted first hash value, the second external electronic device 300 may notify the second external electronic device 300 that the first DICOM file has been forged.

According to an embodiment, the apparatus 400 for verifying forgery & tampering may perform secondary verification on the first DICOM file on which the primary verification has been completed. For example, the apparatus 400 for verifying forgery & tampering may generate a second hash value to be verified using the individual DICOM files to be verified. The apparatus 400 for verifying forgery & tampering may obtain a preregistered second hash value (a hash value of the first DICOM file stored in the DB 100) corresponding to the first issuance number from the verification data of the DB 100. The apparatus 400 for verifying forgery & tampering may perform secondary verification of verifying whether the first DICOM file has been forged by comparing the second hash value to be verified with the preregistered second hash value.

When the second hash value to be verified matches the preregistered second hash value, the apparatus 400 for verifying forgery & tampering may determine that the DICOM file to be verified has not been forged. On the other hand, when the second hash value to be verified does not match the preregistered second hash value, the apparatus 400 for verifying forgery & tampering may determine that the first DICOM file to be verified has been forged.

According to an embodiment, the apparatus 400 for verifying forgery & tampering may provide a result of the verification of the first DICOM file to the second external electronic device 300. For example, when the integrity of the first DICOM file is verified in the primary verification and the secondary verification, the apparatus 400 for verifying forgery & tampering may provide the success of the verification of the first DICOM file to the second external electronic device 300. As another example, when the apparatus 400 for verifying forgery & tampering determines that the first DICOM file has been forged in the primary verification or the secondary verification, the apparatus 400 for verifying forgery & tampering may provide the failure of the verification to the second external electronic device 300.

According to various embodiments, when a plurality of DICOM files (a DICOM file group) of one patient are verified, it is possible to determine whether patient information between the DICOM files is different and display a result of the determination to a verifier to verify whether some DICOM files have been forged. For example, when DICOM files of patient “Yi Sun-shin” are mixed in a DICOM file group of patient “Hong Gil-dong,” it can be indicated that there is patient information of patients “Hong Gil-dong” and “Yi Sun-shin,” making verification easier. That is, it can be verified that an image of patient “Yi Sun-shin,” who is a cancer patient, is added to an image of patient “Hong Gil-dong,” who is a normal person.

Hereinafter, a specific example of verification of forgery of a DICOM file according to an embodiment will be described.

The Military Manpower Administration staff may access a specified second website through the second external electronic device 300 to verify forgery of a DICOM file submitted by a person liable for military service. The second external electronic device 300 may upload the first DICOM file(s) dragged to and dropped in a specific area of the specified second website according to an input of the Military Manpower Administration staff, and request verification of the forgery from the apparatus 400 for verifying forgery & tampering.

First, when the second external electronic device 300 performs primary verification of forgery and the primary verification is passed, secondary verification of forgery may be performed by transmitting an issuance number extracted from the first DICOM file and hash information of the first DICOM file to the apparatus 400 for verifying forgery & tampering.

The apparatus 400 for verifying forgery & tampering may display personal information (e.g., name, gender, and date of birth) confirmed in the process of checking whether the first DICOM file has been forged and thus support the Military Manpower Administration staff to compare the personal information with an identification card independently verified by the Military Manpower Administration staff.

In this way, the apparatus 400 for verifying forgery & tampering according to an embodiment may add forgery verification information to medical data (a DICOM file) that has not been separately subjected to forgery verification, and then support the forgery verification on the basis of the forgery verification information of the medical data, and thus prevent the medical data from being illegally used for purposes such as exemption from military service.

Further, since the apparatus 400 for verifying forgery & tampering according to an embodiment may take much time for forgery verification based on a DICOM file, the DICOM file may be precisely verified in the secondary verification after being verified once in the primary verification by the second external electronic device 300.

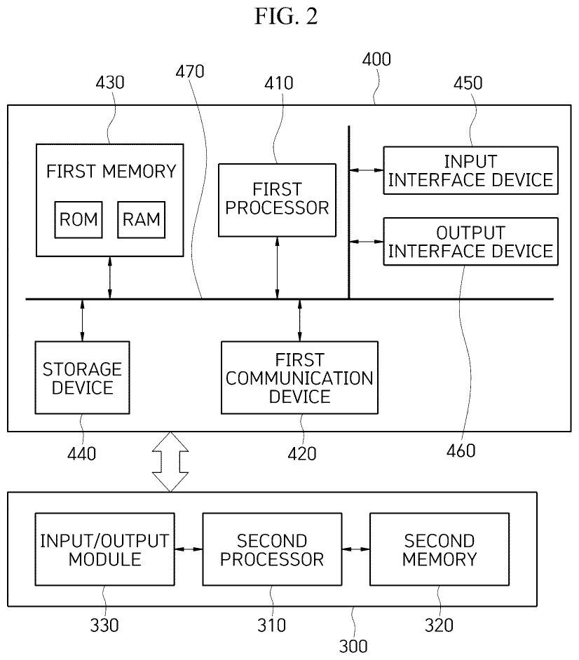

FIG. 2 illustrates a configuration diagram of an apparatus for verifying forgery and tampering of a medical image according to an embodiment.

Referring to FIG. 2, an apparatus 400 for verifying forgery & tampering according to an embodiment may include at least one of a first processor 410, a first memory 430, an input interface device 450, an output interface device 460, and a storage device 440 that communicate through a bus 470. The apparatus 400 for verifying forgery & tampering may further include a first communication device 420 coupled to a network. In an embodiment, some components of the apparatus 400 for verifying forgery & tampering may be omitted or the apparatus 400 for verifying forgery & tampering may further include an additional component. Further, some components of the apparatus 400 for verifying forgery & tampering may be combined to form a single entity but may identically perform functions of the corresponding components prior to combination. For example, the apparatus 400 for verifying forgery & tampering may include a first device that provides a DICOM file issuance service and a second device that provides a DICOM file forgery verification service. In this document, an example in which the apparatus 400 for verifying forgery & tampering provides both the DICOM file issuance service and the DICOM file forgery verification service in a single computing device will be described. However, the present disclosure is not limited thereto.

The first memory 430 and the storage device 440 may include various types of volatile memories or non-volatile memories. For example, the first memory 430 may include a read-only memory (ROM) and a random access memory (RAM). In an embodiment, the first memory 430 may be located inside or outside the first processor 410, and the first memory 430 may be connected to the first processor 410 through various methods that are already known. The first memory 430 may store various data used by at least one component (e.g., the first processor 410) of the apparatus 400 for verifying forgery & tampering. The data may include, for example, input data or output data for software and commands related thereto. For example, the first memory 430 may store at least one instruction and data for providing a DICOM file issuance/forgery verification service.

The first communication device 420 may support the establishment of a communication channel or wireless communication channel between the apparatus 400 for verifying forgery & tampering and other devices (e.g., the first and second external electronic devices 200 and 300) and the performance of communication through the established communication channel. The communication channel may include, for example, at least one communication channel among a local area network (LAN), fiber-to-the-home (FTTH), a digital subscriber line (xDSL), WiBro, a wireless LAN, Wi-Fi, ultra-wideband (UWB), 3^{rd}-generation (3G) communication, 4^{th}-generation (4G) communication, and 5^{th}-generation (5G) communication. The first communication device 420 may communicate using a known communication method such as code division multiple access (CDMA), Global System for Mobile Communications (GSM), wideband code-division multiple access (WCDMA), time-division synchronous code-division multiple access (TD-SCDMA), WiBro, Long-Term Evolution (LTE), EPC, etc.

The first processor 410 may control at least one other component (e.g., a hardware or software component) of the apparatus 400 for verifying forgery & tampering and perform various data processing or operations. The first processor 410 may include, for example, at least one of a central processing unit (CPU), a graphics processing unit (GPU), a microprocessor, an application processor, and an application-specific semiconductor (an application-specific integrated circuit (ASIC) or field programmable gate array (FPGA)) and may have a plurality of cores. The first processor 410 may be a CPU or may be a semiconductor device that executes instructions stored in the first memory 430 or storage device 440.

According to an embodiment, the first processor 410 may obtain a request for issuance of a DICOM file from a user (e.g., medical staff or a patient) through the input interface device 450 or the first communication device 420. The first processor 410 may directly obtain the request for issuance from the user through the input interface device 450 or may obtain the request for issuance from the first external electronic device 200. In an embodiment, the first processor 410 may obtain first patient information (e.g., a resident registration number and patient registration number) together with the request for issuance of the DICOM file. The first processor 410 may further obtain information on a type of the DICOM file for which issuance has been requested (e.g., a department of medicine, an examination date, or examination name).

According to an embodiment, when the first processor 410 obtains the request for issuance, the first processor 410 may search for a DICOM file corresponding to the first patient information among the DICOM files for each patient from the medical DB 100 on the basis of the first patient information (or the first patient information and the information on the type of the DICOM file). Further, when the first processor 410 obtains the request for issuance, the first processor 410 may generate a first issuance number.

According to an embodiment, the first processor 410 may generate forgery verification information on the basis of at least some information of metadata of the searched DICOM file. For example, the first processor 410 may generate a first hash value (forgery verification information) using at least some information of the metadata of the DICOM file and the first issuance number. This information may include, for example, at least one of the first patient information and an issuer of a DICOM file group (e.g., hospital name).

Additionally, the first processor 410 may further use the hash value (third hash value) of the searched individual DICOM file to generate a first hash value for primary verification.

Additionally, the first processor 410 may further use other information (e.g., an issue date) of the searched DICOM file to generate a first hash value for the primary verification.

According to an embodiment, the first processor 410 may transmit a copy of the DICOM file to the first external electronic device 200 (or external storage device 250) and insert the first hash value into a specified tag area of the corresponding copy as forgery verification information. The specified tag area may be, for example, a user-defined tag area of the DICOM file. The first processor 410 may further insert other characteristic information (e.g., an issuance number and issuer information) into the specified tag area as the forgery verification information.

According to an embodiment, the first processor 410 may generate a second hash value on the basis of the copy of the individual DICOM file into which the forgery verification information is inserted. In an embodiment, the first processor 410 may store verification data including a first issuance number and a second hash value in the DB 100 to verify forgery of the issued DICOM file.

According to an embodiment, a DICOM file to be issued may be composed of a plurality of DICOM files included in a DICOM file group. In this case, the first issuance number may include serial numbers of the respective DICOM files and an original number (or unique number) of the DICOM file group. Accordingly, the first processor 410 may calculate first hash values of the respective DICOM files using the serial numbers of the respective DICOM files and the original number and insert the calculated first hash values into the respective DICOM files. In this case, the first hash values of the respective DICOM files may be different from those of other DICOM files in the DICOM file group.

According to an embodiment, when a DICOM file is issued online according to an online request of the first external electronic device 200, the first processor 410 may compress (encrypt) copies of the DICOM files on the basis of a password transmitted from the first external electronic device 200 and transmit the compressed (encrypted) file to the first external electronic device 200. In this case, the first external electronic device 200 may compress the copies of the DICOM files using the password and then store the compressed file in the external storage device 250.

Hereinafter, the verification of forgery will be described.

The second external electronic device 300 according to an embodiment may include an input/output module 330, a second memory 320, and a second processor 310. The input/output module 330 may include an input module such as a keyboard, a display, and a communication module. In an embodiment, the second memory 320 may be located inside or outside the second processor 310, and the second memory 320 may be connected to the second processor 310 through various methods that are already known. The second memory 320 may store various data used by at least one component (e.g., the second processor 310) of the second external electronic device 300. The data may include, for example, input data or output data for software and commands related thereto. For example, the second memory 320 may store at least one instruction and data for primary verification of forgery of a DICOM file. The second processor 310 may control at least one other component (e.g., a hardware or software component) of the second external electronic device 300 and perform various data processing or operations. The second processor 310 may include, for example, at least one of a CPU, a GPU, a microprocessor, an application processor, and an application-specific semiconductor (an ASIC or FPGA) and may have a plurality of cores. The second processor 310 may be a CPU or may be a semiconductor device that executes instructions stored in the second memory 320.

According to an embodiment, in the primary verification process, the first processor 410 may provide a first website related to the primary verification of forgery to the second external electronic device 300. In this case, the second processor 310 of the second external electronic device 300 may access the first website and extract a first hash value and first issuance number from a specified tag area of a first DICOM file through its own web browser. The specified tag area may be at least a part of a user-definable tag area. Further, the second processor 310 may generate a first hash value to be verified on the basis of some information of metadata of the first DICOM file. The second processor 310 may perform the primary verification by comparing the first hash value to be verified with the extracted first hash value. When the first hash value to be verified is found not to match the extracted first hash value as a result of the primary verification, the second processor 310 of the second external electronic device 300 may notify the input/output module 330 that the first DICOM file has been forged.

On the other hand, when the first hash value to be verified matches the extracted first hash value, the second external electronic device 300 (the second processor 310) may perform secondary verification on the first DICOM file. The second external electronic device 300 may generate a second hash value to be verified using the entire first DICOM file to be verified. The second external electronic device 300 may transmit the generated second hash value to the apparatus 400 for verifying forgery & tampering through the input/output module 330.

The first processor 410 may obtain a preregistered second hash value corresponding to the first issuance number from verification data of the DB 100. The first processor 410 may obtain a request for secondary verification of forgery of the first DICOM file from the second external electronic device 300 through the input interface device 450 or the first communication device 420. Upon confirming the request for verification, the first processor 410 may finally verify whether the first DICOM file has been forged through the secondary verification of forgery.

For example, the apparatus 400 for verifying forgery & tampering may perform the secondary verification of whether the first DICOM file has been forged by comparing the second hash value to be verified received from the second external electronic device 300 with the preregistered second hash value in DB 100. When the second hash value to be verified matches the preregistered second hash value in DB 100, the first processor 410 may determine that the first DICOM file for which forgery verification has been requested has not been forged. On the other hand, when the second hash value to be verified does not match the preregistered second hash value in DB 100, the first processor 410 may determine that the first DICOM file for which forgery verification has been requested has been forged.

According to an embodiment, the first processor 410 may provide a result of the secondary verification to a second user (e.g., a verification requester) through the output interface device 460 or the second external electronic device 300.

According to various embodiments, when the secondary verification is also completed, the first processor 410 may provide the personal information (e.g., name, gender, and date of birth) confirmed in the process of checking whether the first DICOM file has been forged to the second external electronic device 300. In this case, the second external electronic device 300 may support the second user to compare the personal information with a submitter's identity in the first DICOM file by outputting the personal information. For example, the first processor 410 may extract the date of birth, name, and gender among the patient information from the metadata of the first DICOM file and output the extracted patient information through the second external electronic device 300. Accordingly, the second external electronic device 300 may support the second user to compare input patient information (or the identity on the ID or certificate of the submitter) with the extracted patient information. As another example, the first processor 410 may check the first issuance number in the metadata of the first DICOM file and obtain a resident registration number corresponding to the first issuance number of the first DICOM file from the DB 100. The first processor 410 may compare the obtained resident registration number with an input resident registration number.

In various embodiments, when preregistered patient information does not match the input patient information, the first processor 410 or the second user may determine that the first DICOM file has been forged.

In this way, the apparatus 400 for verifying forgery & tampering according to an embodiment may add forgery verification information to medical data (a DICOM file) that has not been separately subjected to forgery verification, and then support the forgery verification on the basis of the forgery verification information of the medical data, and thus prevent the medical data from being illegally used for purposes such as exemption from military service.

Further, because the apparatus 400 for verifying forgery & tampering according to an embodiment may take much time for forgery verification based on the entire DICOM file, the DICOM file may be precisely verified in the secondary verification after being verified once in the primary verification by the second external electronic device 300.

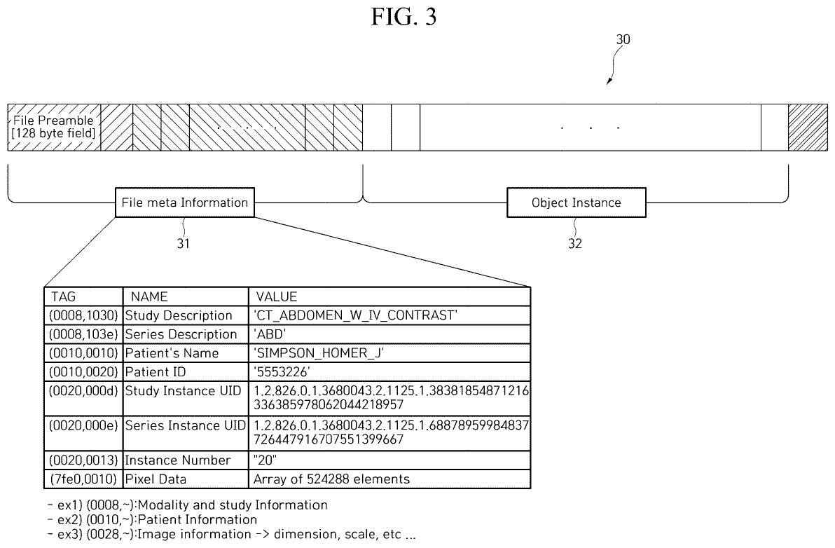

FIG. 3 illustrates an example in which a DICOM file according to an embodiment is used.

Referring to FIG. 3, a DICOM file 30 according to the embodiment may include a file metadata area 31 and an object instance area 32. The apparatus 400 for verifying forgery & tampering may insert a first hash value into a specified tag area in the file metadata area 31. The apparatus 400 for verifying forgery & tampering may further insert other characteristic information (e.g., an issuance number and issuer information) into the specified tag area. The specified tag area may be, for example, a user-defined tag area (e.g., Ox0011) of the DICOM file.

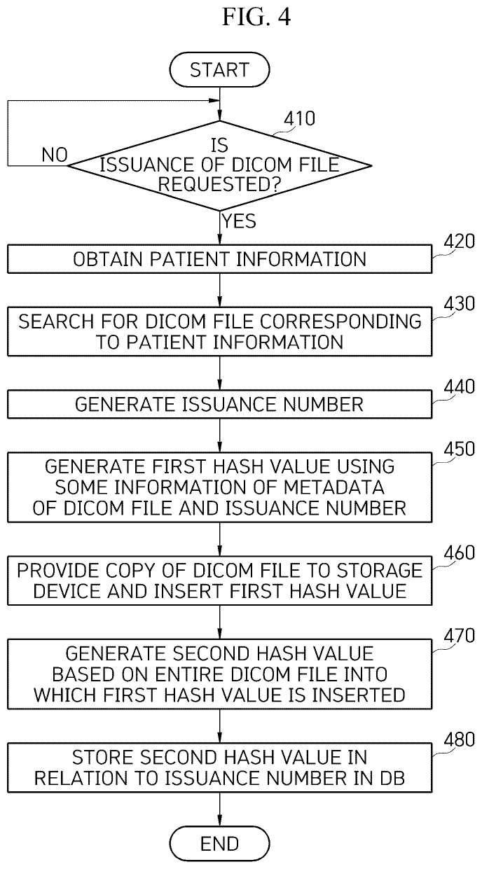

FIG. 4 illustrates a flowchart of a method of verifying forgery and tampering of a medical image according to an embodiment.

Referring to FIG. 4, in operation 410, the apparatus 400 for verifying forgery & tampering may obtain a request for issuance of a DICOM file from the first external electronic device 200.

In operation 420, the apparatus 400 for verifying forgery & tampering may obtain patient information related to the request for issuance from the first external electronic device 200. For example, the apparatus 400 for verifying forgery & tampering may obtain patient information included in a request for verification of forgery of the DICOM file.

In operation 430, the apparatus 400 for verifying forgery & tampering may search for a DICOM file corresponding to the obtained patient information from the DB 100.

In operation 440, the apparatus 400 for verifying forgery & tampering may generate an issuance number related to the issuance of the DICOM file. When the DICOM file belongs to a DICOM file group including a plurality of DICOM files, the issuance number may include an original number assigned to one DICOM file group and serial numbers of the respective DICOM files.

In operation 450, the apparatus 400 for verifying forgery & tampering may generate a first hash value using some information of metadata of the DICOM file and the issuance number.

In operation 460, the apparatus 400 for verifying forgery & tampering may provide a copy of the DICOM file to the first external electronic device 200 to insert the first hash value into a specified tag area of the copy. The first hash value may be used for primary verification of forgery of the copy of the DICOM file.

In operation 470, the apparatus 400 for verifying forgery & tampering may generate a second hash value for an individual file using copies of the individual DICOM files into which the first hash value has been inserted.

In operation 480, the apparatus 400 for verifying forgery & tampering may store the second hash value related to the issuance number as verification data of the copy of the DICOM file in the DB 100. The first hash value may be used for secondary verification of forgery of the copy of the DICOM file.

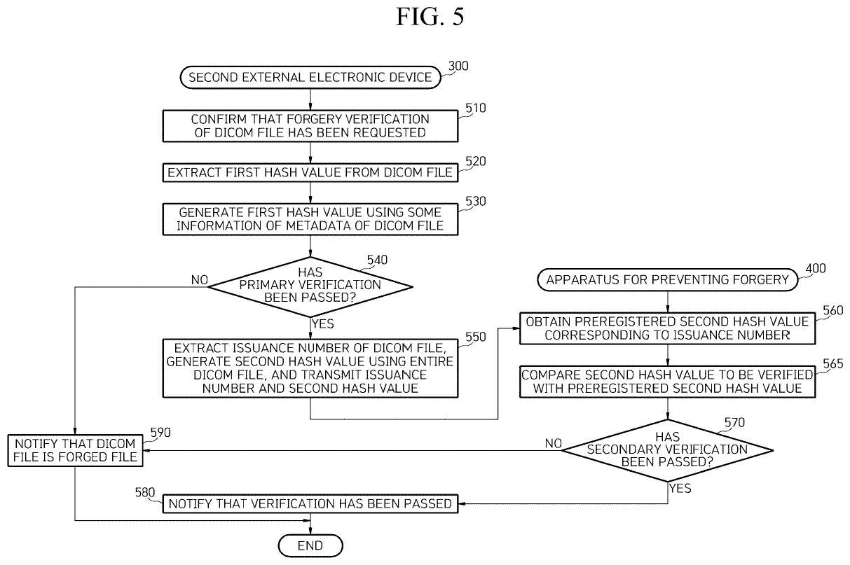

FIG. 5 illustrates a flowchart of a method of verifying forgery of a medical image according to an embodiment.

Referring to FIG. 5, in operation 510, the second external electronic device 300 may confirm that forgery verification of a DICOM file has been requested.

In operation 520, the second external electronic device 300 may extract a first hash value from the DICOM file.

In operation 530, the second external electronic device 300 may generate a first hash value using some information of metadata of the DICOM file.

In operation 540, the second external electronic device 300 may check whether the generated first hash value matches the extracted first hash value and thus passes primary verification.

In operation 540, when it is checked that the primary verification has been passed, the second external electronic device 300 may generate a second hash value (second hash value to be verified) of the individual DICOM file in operation 550.

In operation 550, the second external electronic device 300 extracts an issuance number from a DICOM file to be verified, calculates a second hash value of the DICOM file, and transmits the issuance number and the second hash value to the apparatus 400 for verifying forgery & tampering.

In operation 560, when the apparatus 400 for verifying forgery & tampering obtains the issuance number and the second hash value to be verified from the second external electronic device 300, the apparatus 400 for verifying forgery & tampering may obtain a preregistered second hash value corresponding to the issuance number from the DB 100.

In operation 565, the apparatus 400 for verifying forgery & tampering may check whether the second hash value to be verified received from the second external electronic device 300 matches the preregistered second hash value.

In operation 570, the apparatus 400 for verifying forgery & tampering may check whether secondary verification has been passed on the basis of whether the second hash value to be verified matches the preregistered second hash value.

When it is checked in operation 570 that the secondary verification has been passed, the apparatus 400 for verifying forgery & tampering may notify the second external electronic device 300 in operation 580 that the DICOM file has not been forged.

When the second external electronic device 300 confirms in operation 540 that the primary verification has not been passed, the second external electronic device 300 may notify itself in operation 590 that the DICOM file is a forged file. Alternatively, when the apparatus 400 for verifying forgery & tampering confirms in operation 580 that the secondary verification has not been passed, the apparatus 400 for verifying forgery & tampering may notify the second external electronic device 300 in operation 590 that the DICOM file is a forged file.

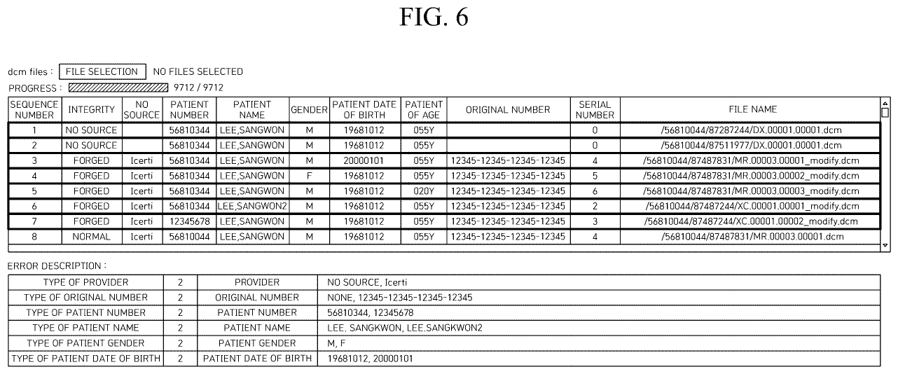

FIG. 6 illustrates an example in which a result of verification according to an embodiment is provided.

Referring to FIG. 6, the second external electronic device 300 according to an embodiment may check whether to match the provider (issuing office), patient number, patient name, gender, patient date of birth, original number, serial number, and file name of each of the remaining DICOM files, excluding 7 DICOM files out of a total of 9,721 DICOM files included in one DICOM file group, in the primary verification process. When the second external electronic device 300 confirms that the provider and original number are not included in the result of the primary verification in sequence numbers 1 and 2, the second external electronic device 300 may notify of the forgery in different colors for the forged DICOM files. Therefore, additionally, the second user may visually compare the personal information included in the result of the verification of the DICOM file with a submitter's identity of the DICOM file to verify whether the submitter's identity has been stolen. Further, the second external electronic device 300 according to an embodiment may provide the organized content of all the files provided as an error history. When there are two or more types of DICOM files for which verification is requested, it can be confirmed that the DICOM files are not image data (DICOM files) of one person. Accordingly, the second user may visually confirm that image data (DICOM file) of another person is included in a file to be verified.

According to various embodiments disclosed in this document, forgery of medical images can be verified using hash values based on metadata of the medical images. In addition, various effects that can be directly or indirectly identified through this document can be provided.

It should be understood that various embodiments disclosed in this document and terms used herein are not intended to limit the technical features described in this document to specific embodiments, but rather to encompass various modifications, equivalents, or alternatives of the corresponding embodiments. With respect to the description of the drawings, similar reference numerals may be used for similar or related components. A singular form of a noun corresponding to an item may include one item or a plurality of items, unless the context clearly indicates otherwise. In this document, each of expressions such as “A or B,” “at least one of A and B,” “at least one of A or B,” “A, B, or C,” “at least one of A, B, and C,” and “at least one of A, B, or C” may include any one of the items listed together in these expressions, or all possible combinations of the listed items. Terms such as “first” or “second” may be used merely to distinguish one component from another component, and do not limit the components in any other respect (e.g., importance or order). When one component (e.g., a first component) is referred to as being “coupled” or “connected” to another component (e.g., a second component), with or without the terms “functionally” or “communicatively,” it means that the one component may be connected to the other component directly (e.g., wired), wirelessly, or through a third component.

The term “module” used in this document may include a unit composed of hardware, software, or firmware and, for example, may be used interchangeably with a term such as “logic,” “logic block,” “part,” or “circuit.” The module may be an integrally constituted part or a minimum unit or a part of the part that performs one or more functions. For example, according to an embodiment, the module may be configured in the form of an ASIC.

Various embodiments disclosed in this document may be implemented as software (e.g., a program) including one or more instructions stored in a storage medium (e.g., a built-in memory or external memory) readable by a machine (e.g., an apparatus for verifying forgery and tampering). For example, a processor (e.g., the first processor 410) of the machine (e.g., the apparatus 400 for verifying forgery & tampering) may call at least one instruction among the one or more instructions stored from a storage medium and execute the instruction. This enables the machine to operate to perform at least one function according to the at least one instruction. The one or more instructions may include code generated by a compiler or code executable by an interpreter. The machine-readable storage medium may be provided in the form of a non-transitory storage medium. Here, “non-transitory” simply means that the storage medium is a tangible device and does not include signals (e.g. electromagnetic waves), and the term does not distinguish that data is semi-permanently or temporarily stored in the storage medium.

According to an embodiment, the methods according to various embodiments disclosed in this specification may be provided by being included in computer program products. The computer program products may be traded between sellers and buyers as commodities. The computer program products may be distributed in the form of a machine-readable storage medium (e.g., a compact disc read only memory (CD-ROM)) or may be distributed online (e.g., by download or upload) through an application store (e.g., Play Store™) or directly between two user devices (e.g., smartphones). In the case of online distribution, at least some of the computer program products may be temporarily stored or temporarily generated in a machine-readable storage medium, such as a memory of a server of a manufacturer, a server of an application store, or a relay server.

The components according to various embodiments disclosed in this document may be implemented in the form of software or hardware such as a digital signal processor (DSP), an FPGA, or an ASIC, and may perform predetermined roles. The components are not limited to software or hardware. Each component may be included in a recording medium that may address the component or may be formed to be executed by at least one processor. Examples of the components may include components such as software components, object-oriented software components, class components, and task components, processes, functions, properties, procedures, subroutines, segments in program codes, drivers, firmware, microcode, circuits, data, DBs, data structures, tables, arrays, and parameters.

According to various embodiments, each of the components (e.g., the module or the program) may include a singular or a plurality of entities. According to various embodiments, one or more of the above-described components or operations may be omitted, or one or more other components or operations may be added. Alternatively or additionally, the plurality of components (e.g., the module or the program) may be integrated into a single component. In this case, the integrated component may perform one or more functions of each of the plurality of components identically or similarly to those performed by the corresponding component among the plurality of components prior to the integration. According to various embodiments, the operations performed by the module, the program, or other component may be performed sequentially, in parallel, repetitively, or heuristically, or one or more of the operations may be performed in a different order or may be omitted, or other operations may be added.

What is claimed is:

1. An apparatus for verifying forgery and tampering of a medical image, comprising:
a memory configured to store Digital Imaging and Communications in Medicine (DICOM) files for each patient; and
a processor functionally connected to the memory,
wherein the processor is configured to:
when an issuance request of a DICOM file is received, search for a DICOM file corresponding to the request among DICOM files for each patient;
generate an issuance number related to the issuance;
extract some information from metadata of the searched DICOM file;
generate a first hash value using the some information and the issuance number; and
upon providing a copy of the DICOM file to an external storage device, insert the first hash value as forgery verification information into a specified tag area of the copy.

2. The apparatus of claim 1, wherein the processor is configured to:
generate a second hash value for the copy of the DICOM file; and
store the second hash value in the memory in relation to the issuance number.

3. The apparatus of claim 1, wherein the some information includes at least one of patient information corresponding to the request and an issuer of the searched DICOM file.

4. The apparatus of claim 2, wherein the processor is configured to: calculate a second hash value for the searched individual DICOM file; and
calculate the first hash value on the basis of the second hash value, the issuance number, and the some information.

5. The apparatus of claim 1, wherein when a plurality of DICOM files including the DICOM file corresponding to the request,
the issuance number includes serial numbers of the respective DICOM files and an original number of the plurality of DICOM files, and
the processor calculates first hash values of the respective DICOM files on the basis of the original number and the serial numbers of the respective DICOM files to provide the first hash values as the first hash value.

6. The apparatus of claim 1, wherein the processor further obtains a password for compression from a requester together with the request, compresses (encrypts) the copy of the DICOM file using the obtained password, and stores the compressed copy in the external storage device.

7. An apparatus for verifying forgery and tampering of a medical image, comprising:
a memory configured to store verification data including issuance numbers of Digital Imaging and Communications in Medicine (DICOM) files for each patient and hash values of each DICOM files; and
a processor functionally connected to the memory,
wherein the processor is configured to:
when obtaining a request for verification of a first DICOM file stored in an external storage device, extract a first hash value and first issuance number from a specified tag area of the first DICOM file;
generate a first hash value to be verified using some information of metadata of the first DICOM file; and
perform primary verification of whether the first DICOM file has been forged by comparing the extracted first hash value with the first hash value to be verified.

8. The apparatus of claim 7, wherein the processor is configured to:
generate a second hash value to be verified for the first DICOM file;
obtain a preregistered second hash value of a DICOM file corresponding to the first issuance number from the verification data; and
perform secondary verification of whether the first DICOM file has been forged by comparing the second hash value to be verified with the preregistered second hash value.

9. The apparatus of claim 8, wherein the first DICOM file is one of a plurality of DICOM files included in a first DICOM file group,
the first issuance number includes serial numbers of the each DICOM files and an original number of the first DICOM file group, and
the processor obtains the preregistered second hash value corresponding to the original number and the serial numbers of the respective DICOM files from the verification data.

10. The apparatus of claim 7, wherein the processor obtains personal information together with the request for verification and verifies whether the first DICOM file has been forged by comparing the obtained personal information with the patient information corresponding to the first issuance number during or before the primary verification.
